(19) United States
(12) Patent Application Publication
(10) Pub. No.: US 2023/0335282 A1
(43) Pub. Date: Oct. 19, 2023

(54) DEVICE AND METHOD FOR DETECTING SICKLE CELL DISEASE USING DEEP TRANSFER LEARNING

(71) Applicant: Morgan State University, Baltimore, MD (US)

(72) Inventor: MD Mahmudur Rahman, Ellicott City, MD (US)

(21) Appl. No.: 18/303,170

(22) Filed: Apr. 19, 2023

Related U.S. Application Data
(60) Provisional application No. 63/332,646, filed on Apr. 19, 2022.

(51) Int. Cl. G16H 50/20; G16H 30/40; G16H 50/70; G06T 7/00

(52) U.S. Cl. CPC G16H 50/20; G16H 30/40; G16H 50/70; G06T 7/0014; G06T 2207/30024

(57) ABSTRACT

A content-based image retrieval (CBIR) system that is applied to blood smear images diagnoses whether sickle cell disease is present. Deep Learning (DL) based Convolutional Neural Networks (CNNs) are designed to recognize visual patterns directly from image pixels with minimal preprocessing. Using pre-trained CNNs as a feature extractor provides an alternative to the handcrafted features that are not manually engineered from raw pixel data in general machine learning (ML) classifiers. The invention relies on extracting features from blood smear images by applying Deep Transfer Learning with three pre-trained models: ResNet-50, Inception V3, and VGG16 using Python with OpenCV and Keras libraries.

### FIELD OF THE INVENTION

The invention relates to methods for the detection of sickle cell disease.

### BACKGROUND OF THE INVENTION

Generally, sickle cell disease (SCD) detection is expensive, inaccurate, and hard to conduct. There is a need for the application of Image Processing and Machine Learning (ML) to predict sickle cell types in blood smear accurately and effectively. U.S. Pat. Nos. 10,552,663, 10,255,693 relate to classification of a more general set of cells and particles for analyzing digital microscopy (cytology) images. An ML-based classification system is applied in U.S. Patent Application No. 20180211380 to biological samples using automated image analysis. U.S. Pat. No. 10,573,003 determines disease class scores for patient tissue using a points-of-interest technique.

### SUMMARY OF THE INVENTION

The present invention relates to a content-based image retrieval (CBIR) system that is applied to blood smear images and diagnoses whether sickle cell disease is present. Deep Learning (DL) based Convolutional Neural Networks (CNNs) are designed to recognize visual patterns directly from image pixels with minimal preprocessing. Using a pre-trained CNN as a feature extractor also provides an alternative to the handcrafted features that are not manually engineered from raw pixel data in general ML classifiers. This invention relies on extracting such features from blood smear images by applying Deep Transfer Learning with pre-trained models. According to non-limiting but preferred embodiments, three pre-trained models are used: ResNet-50, Inception V3, and VGG16 using Python with OpenCV and Keras libraries.

This innovation can be implemented through a software application that allows for a patient's blood smear image to be compared to an image from reference datasets. The software will allow for a doctor to choose the model that will best diagnose sickle cell with a high level of confidence. Algorithms and other processes are incorporated into this software and allows for scores to be calculated that indicate the possibility of carrying sickle cell disease.

Accordingly, there is provided according to an embodiment of the invention an automated method for diagnosing sickle cell disease type from a blood smear image, comprising: receiving at a processor of a diagnosing system computer a digital query image of a blood smear from a data capture device; comparing at said processor said digital query image to a plurality of digital images in a database, wherein said database comprises digital blood smear images of pathologically confirmed types of sickle cell disease; selecting at said processor a plurality of said pathologically confirmed digital images from said database that have a designated similarity to said digital query image; and causing said processor to display to a user the probabilities that said digital query image displays a blood smear having a pathology matching each of a plurality of sickle cell disease types.

According to various further embodiments of the invention, there may be provided one or more of the following:
causing said processor to display said plurality of pathologically confirmed digital images to said user;
applying at said processor a deep feature extraction to said digital query image to generate a feature vector quantifying contents of the digital query image;
using at said processor a plurality of pretrained Convolutional Neural Networks feature vectors to generate a combined feature vector;
applying at said processor a classification to said feature vector as one of multiple types of sickle cell disease;
using both Logistical Regression and Support Vector Classifier processes; and/or
causing said processor to select said plurality of said pathologically confirmed digital images based on a distance measure between a feature vector of said digital query image and said plurality of pathologically confirmed digital images.

According to other embodiments of the invention, there may be provided a system for the automated diagnosing of a sickle cell disease type from a blood smear image, comprising a memory and a processor in data communication with said memory, the memory having computer executable instructions stored thereon configured to be executed by the processor to cause the system to:
receive a digital query image of a blood smear from a data capture device;
compare at said processor said digital query image to a plurality of digital images in a database, wherein said database comprises digital images of blood smears with pathologically confirmed types of sickle cell disease;
select a plurality of said pathologically confirmed digital images from said database that have a designated similarity to said digital query image; and
display to a user, the probabilities that said digital query image displays a blood smear having a pathology matching each of a plurality of sickle cell disease types.

Additionally, according to various further embodiments of the invention, said computer executable instructions may be further configured to
cause said processor to display said plurality of pathologically confirmed digital images to said user;
compare said digital query image to the plurality of digital images are further configured to apply a deep feature extraction to said digital query image to generate a feature vector quantifying contents of the digital query image;
apply a deep feature extraction to said digital query image and to use a plurality of pretrained Convolutional Neural Networks feature vectors to generate a combined feature vector;
compare said digital query image to the plurality of digital images and to apply a classification to said feature vector as one of multiple types of sickle cell disease;
apply a classification to said feature vector and to use both Logistical Regression and Support Vector Classifier processes; and/or
to select said plurality of said pathologically confirmed digital images based on a distance measure between a feature vector of said digital query image and said plurality of pathologically confirmed digital images.

According to still other embodiments of the invention, there may be provided a non-transitory computer-readable medium having stored thereon one or more code sections each comprising a plurality of instructions executable by one or more processors, the instructions configured to cause the one or more processors to perform the actions of an automated method for diagnosing a sickle cell disease type, the actions of the method comprising the steps of:
receiving a digital query image of a blood smear from a data capture device;
comparing said digital query image to a plurality of digital images in a database, wherein said database comprises digital images of blood smears with pathologically confirmed types of sickle cell disease;
selecting a plurality of said pathologically confirmed digital images from said database that have a designated similarity to said digital query image; and
displaying to a user, the probabilities that said digital query image displays a blood smear having a pathology matching each of a plurality of sickle cell disease types.

According to still further embodiments, the processor may display said plurality of pathologically confirmed digital images to said user and/or select said plurality of said pathologically confirmed digital images based on a distance measure between a feature vector of said digital query image and said plurality of pathologically confirmed digital images.

### BRIEF DESCRIPTION OF DRAWINGS

FIG. 1 provides a schematic view of a system for aiding in the diagnosis of a sickle cell disease through digital image processing in accordance with certain aspects of an embodiment of the invention.

FIG. 2 is a schematic diagram of a workflow for training and classifying prior pathologically confirmed sickle cell blood smear images for use with system 100.

FIG. 3 is a schematic diagram of a CNN that may be implemented by a feature extraction module of the system of FIG. 1.

FIG. 4 is a schematic diagram of a feature vector classification process that may be implemented by a classification module of the system of FIG. 1.

FIG. 5 depicts a computer interface that displays blood smear images, input models and results from applying specific neural networks/classifiers with listed probabilities.

FIG. 6 is a schematic view of an exemplary computer system suitable for implementing the methods described herein.

### DETAILED DESCRIPTION OF THE INVENTION

The invention summarized above may be better understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Systems and methods configured in accordance with certain aspects of the invention provide a content-based image retrieval (CBIR) system that serves as a diagnostic aid that provides a set of blood smear images of pathologically confirmed sickle cell disease, which are of high similarity to an unknown new case in question, along with the diagnostic profiles of the confirmed images. While such systems and methods are not intended per se as a replacement for a physician by predicting the disease state of a particular case, such systems and methods may be used as a diagnostic aid for both general practitioners and less practiced physicians in making such diagnoses.

Systems and method configured in accordance with certain aspects of the invention may be multi-disciplinary in nature, as they may combine techniques from several fields, such as image processing, computer vision, information retrieval, deep learning, and data mining.

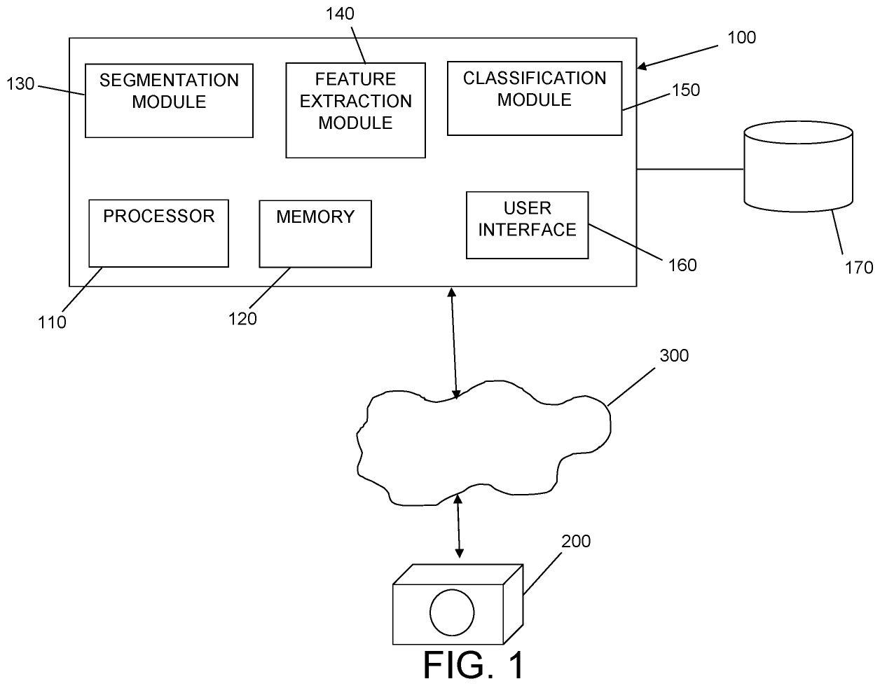

FIG. 1 provides a schematic view of a system for aiding in the diagnosis of a sickle cell disease through digital image processing in accordance with certain aspects of an embodiment of the invention. As shown in FIG. 1, system 100 includes a processor 110, memory 120, image segmentation module 130, feature extraction module 140, classification module 150, user interface 160, and database 170 containing blood smear images of pathologically confirmed sickle cell disease. The functions of each of the foregoing are discussed in greater detail below. However, by way of summary, system 100 is preferably accessible via a medical practitioner, to enable that medical practitioner to transmit a digital image of a patient's blood smear that is captured using an image capture device 200, such as a digital microscope camera, or such other digital image capture device as may be apparent to those skilled in the art, through a wide area network 300 such as the Internet, which digital image may be used by system 100 as a query image to search for similar images in database 170, and thus similar pathological diagnosis for similar blood smears of other patients. The medical practitioner preferably engages system 100 through user interface 160 (discussed in greater detail below) and may have the option of using image segmentation module 130 to segment the image for detecting a sickle cell as a minimum bounding box (MBR) or use the entire image with background information. Deep features of the blood cells in the query image are then extracted from the query image by feature extraction module 140. Next, system 100 uses classification module 150 to perform the task of classification and retrieves relevant images of past cases present in database 170. In certain optimal configurations, the medical practitioner using user interface 160 may select differing CNN selections which may be fused using a regression analysis, and an ensemble of classification models can be used for the final image classification. Further, the medical practitioner may optionally select different similarity measures and feature fusion approaches in the deep feature spaces of database and query images for both flexibility and effectiveness. Following the classification and retrieval of relevant images, such retrieved blood smear images containing pathologically confirmed sickle cell disease that have been automatically determined as similar to the query image of the patient's blood smear may then be transmitted through network 300 to the medical practitioner for display on their local device to aid in making a diagnosis of the patient's condition.

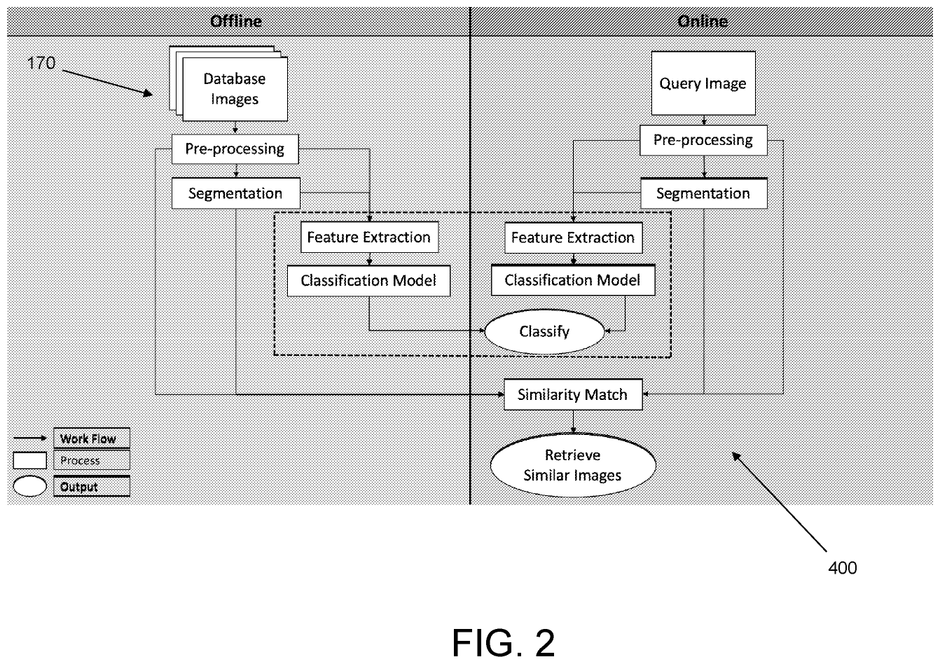

As shown in the schematic view of FIG. 2, database 170 of blood smear images with pathologically confirmed sickle cell disease may be formed, maintained, and updated in a system 400 that employs both offline and online phases. During offline processing, images of blood smears of known pathology are trained for classification and indexed in database 170. During such offline processing, system 100 may first pre-process each image that is to be included in database 170 by resizing each such image for the respective CNN approaches that are to be employed by system 100. Using the transfer learning approach, the deep features of the confirmed images are extracted by passing them through the CNNs that are without a classification head. The results obtained after this stage are the features that would have been passed to the classification layer. Classification models, such as Logistic Regression and Support Vector Machine (SVM) (the methods of which are known to those skilled in the art) are then built on top of the extracted features as these bottleneck features learned by the CNNs are quite distinct and specific to each image.

The dataset used consisted of 453 sickle cell blood smear images, including 157 images associated with Hemoglobin S disease type (SS), 143 images associated with Hemoglobin C diseases type (SC), and 153 images associated with Hemoglobin Beta Thalassemia (S Beta+). The images were obtained from Hematology Day Care, Ibadan, Department of Information Technology-Universitá Degli Studi di Milano, as well as Google Images. The data was split into a test subset and a training subset. The training subset represented 70% of the data, while the testing subset represented 30% of the data. The multi-class labelling was recorded for each image in the dataset, and it was saved in an Excel file. The training subset data and corresponding image multi-class labels were utilized in training the proposed classification model.

The models that were used in image classification and image retrieval were: ResNet 50, VGG 16 and Inception V3. These models were compared, to identify which of the models has the highest score of prediction and accuracy.

Multi-Classification. The testing subset data was used in evaluating the performance of the trained classification model with its corresponding multi-class labels serving as the evaluation ground-truth. The labels were one-hot encoded into a vector representation of the 3 disease classes. During the training, the deep pretrained model layers were frozen to avoid modifying the tuned neurons from the previous training. Freezing these layers means the knowledge gained from the previous training is preserved while the newly added fully connected classification head is trained. The model receives an image input of size 224 by 224 for ResNet-50 and VGG-16 models and 299 by 299 for Inception V3 model. These images were passed through the pre-trained model layers resulting in feature outputs with different dimensions. The feature output obtained from the pretrained layers represents the information identified by the pretrained deep neural models. This feature output was passed through the newly added fully connected classification head to give an output inference of image input multiclass vector. i.e., [1, 0, 0]. The process of fine tuning the classification head neurons to make the correct output inferences is referred to as the model training. The model was trained for 15 epochs and Adam Optimizer. The trained model was evaluated by computing the confusion matrix for the results given for the test subset data. A precision—recall curve was also generated to evaluate the effect of the three different pretrained models on the classification.

Unlike the binary classification method, precision-recall score is calculated for every class in the multi-classification method. So, this means that for every class, there is a calculation of the F1-score. Precision tells what fraction of predictions as a positive class were positive and the formula for precision is

$$\frac{T P}{( T P + F P )} ,$$

where TP is the true positive, while FP is the false positive.

Recall tells the correctly predicted fraction by the classifier, and the formula is

$$\frac{T N}{( T N + F P )} ,$$

where

TN is the Truth Negative.

The true positive is the outcome where the model correctly predicts the positive class. The true negative is the outcome where the model incorrectly predicts the positive class. The false positive is the outcome where the model correctively predicts the negative class. The false negative is the outcome where the model incorrectly predicts the negative class. The F1 score is basically the combination of the precision and the recall, and its formula is:

$${F}_{1} - Score = 2 \times \frac{P r e c i s i o n \times R e c a l l}{P r e c i s i o n + R e c a l l} = \frac{2 T P}{2 T P + F P + F N}$$

The TP, TN, FP, and FN (false negative) for each class (SS, SC, S Beta+), may be calculated as depicted in the formulas below:

$$\begin{matrix}SS {F}_{1} - Score = 2 \times \frac{P r e c i s i o n \times R e c a l l}{P r e c i s i o n + R e c a l l} = \frac{2 T P}{2 T P + F P + F N} \\ SC {F}_{1} - Score = 2 \times \frac{P r e c i s i o n \times R e c a l l}{P r e c i s i o n + R e c a l l} = \frac{2 T P}{2 T P + F P + F N} \\ SB {F}_{1} - Score = 2 \times \frac{P r e c i s i o n \times R e c a l l}{P r e c i s i o n + R e c a l l} = \frac{2 T P}{2 T P + F P + F N}\end{matrix}$$

Retrieval. The first phase of this methodology involved computing the features from the pre-trained models for the dataset training subset images and saving them in a database. For each image, the feature extraction involved obtaining the feature output from the layer before the classification layer of the classification model. The next phase involves applying the same feature extraction method to a query image. The query image features are compared with each image features in the database by measuring the extent of similarity. The top N most similar images are returned as the result. Vantage point tree is used to partition the data points for the search query and the stored features.

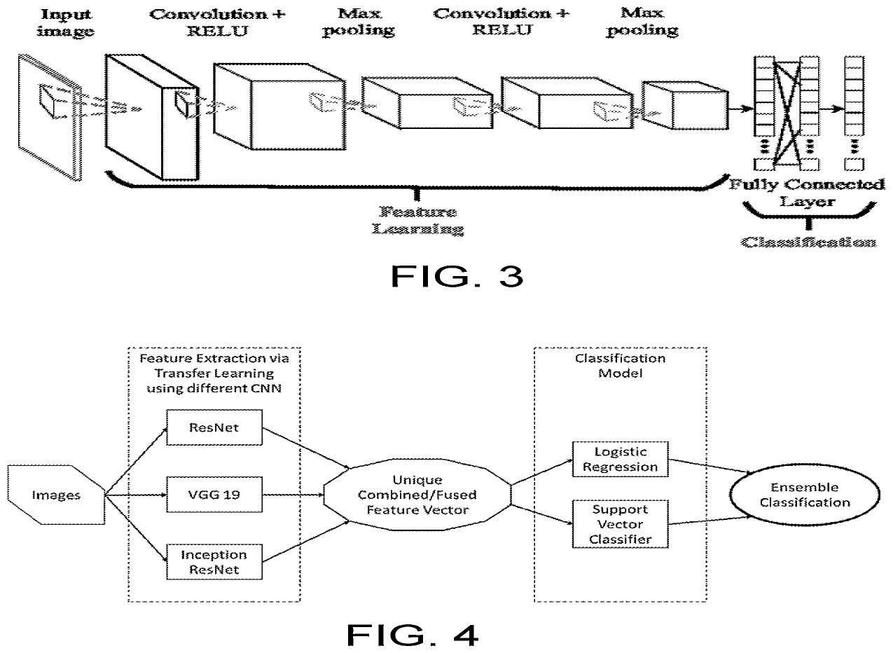

Euclidean Distance measure is used to compute the similarity between the search query and stored features. The distance between the two points (feature vector of the stored data and feature vector of the query data) is checked. If a specific point from the stored dataset (representing an image's features) is the closest to that of a precise query image, the features of the image are considered to be similar to that of the query image. FIG. 3 illustrates distance between feature x and feature y. The formula for checking the distance is:

EUD=\sqrt{Σᵢ₌₁ⁿ(Xᵢ−Yᵢ)²}

The precision and recall are calculated in this methodology, where Precision is number relevant to the query building retrieved images for the total number of retrieved images and System Recall has to do with the number relevant to the query building retrieved images for the number of identical query building retrieved images.

VGG 16 model's F1 score for Hemoglobins Beta, C, and S were 0.87, 0.69, and 0.67, respectively. VGG 16 accurately predicted Hemoglobin Beta up to 87%, Hemoglobin C up to 69%, and Hemoglobin S up to 67%. Inception-V3 model accurately predicted Hemoglobin beta up to 83%, Hemoglobin C up to 82%, and Hemoglobin S up to 81%. ResNet 50 model accurately predicted hemoglobin Beta up to 95%, Hemoglobin C up to 86%, and Hemoglobin S up 78%. Overall, VGG 16 classified the tested images with an accuracy of 72%, Inception-V3 with an accuracy of 81%, while ResNet 50 classified the images with an accuracy of 87%. In short, the models satisfactorily predicted the multi-image classification (above 50%).

To implement the feature learning methods, a distributed deep-learning library may be used, which may be written in computer languages such as Python, Java and Scala, and integrated with Hadoop and Spark.

Classification module 170 may be used to classify the images in multiple sickle cell disease types. In a particular preferred configuration, systems and methods employing aspects of the invention may incorporate an ensemble of classification models, which may include (by way of non-limiting example) a Logistical Regression (LR) model and Support Vector Classifier (SVC) model, each trained on each individual or fused feature vectors with different feature combinations produced by feature extraction module 140, as shown in the schematic view of FIG. 4. Preferably, a user may employ user interface 160 to choose any combination of classifiers for ensemble learning. Ensemble learning involves taking multiple classifiers and aggregating them into a single meta-classifier. By averaging multiple machine learning models together, we may outperform (i.e., achieve higher accuracy) by using just a single model chosen at random. In a particular exemplary configuration, multiple networks were trained and then asked to return the probabilities for each class label given an input data point. Such probabilities are averaged together, and the final classification is obtained. By averaging multiple machine learning models together, higher accuracy may be achieved by using just a single model chosen at random.

Similarity matching is an essential final processing step employed by system 100 and is used to select and display to the medical practitioner via user interface 160 probability classifications for the queried patient blood smear image, preferably including a calculated probability of each classification (i.e., disease type, if any) for the queried image, and preferably including images of the most similar images in database 170 to the queried image (as discussed in greater detail below). For a given query image captured by image capture device 200 and transmitted to system 100, a search is made on the images in database 170 based on the deep features representing each blood smear image. The difference between the feature vector of the query image (patient blood smear) and the feature vectors of blood smears of reference images in database 170 is preferably calculated based on different distance measures, such as Euclidean, Manhattan, and Cosine methods (which methods are known to those skilled in the art) to compute the similarity between the query image and the database. Current CAD schemes using CBIR approaches typically use the k-nearest neighbor type searching method, which involves searching from the k most similar reference ROIs (i.e., blood smears) to the queried ROI (i.e., patient blood smears). The smaller the difference (i.e., “distance”), the higher the computed “similarity” level is between the two compared ROIs. The searching and retrieval result of the CBIR algorithm depends on the effectiveness of the distance metrics to measure the similarity level among the selected images. Preferably, the query-specific adaptive similarity fusion approach set forth herein effectively exploits the online blood smear classification information and adjusts the feature weights accordingly in a dynamic fashion.

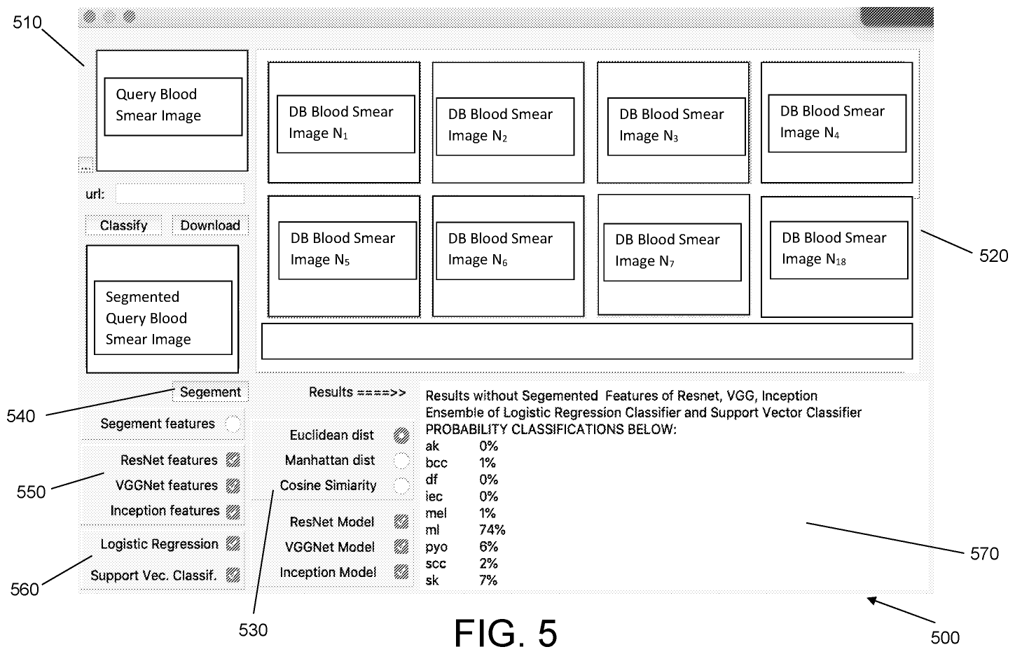

Next, FIG. 5 shows a display that may be presented to a user, such as the medical practitioner that transmitted the query image to system 100, by user interface 160 of system 100. The user interface 500 primarily consists of a query panel 510 to display the query image (which can be selected either from an URL or browsed through a folder), and a display panel 520 to show the most similar images to the query image based on selecting a distance measure 530, such as Euclidean, Manhattan, and Cosine to perform similarity matching. In addition, the interface also provides the options for segmenting at 540 the query image for sickle cell disease detection, and also options for selecting different combinations of deep features 550 and classification model 560.

Hence, a combination of CN Ns can be selected to fuse features for a query image and a late fusion of classification probabilities can be made with checkbox selections of either Logistic Regression Classification or SVM Classifiers. The classification result as probabilistic outputs of different categories are displayed in Probability Classifications window 570 as percentages. Overall, the interface presented to the user is very user friendly and flexible for the user where he/she can perform both classification and retrieval by selecting from a number of options.

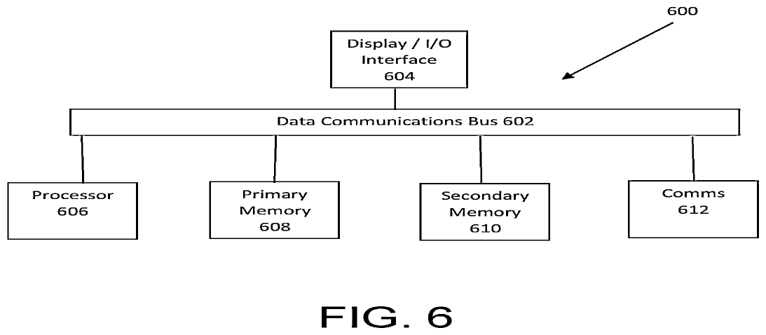

Next, FIG. 6 shows an exemplary computer system 600 suitable for implementing the methods described herein. Those skilled in the art will recognize that system 100 for aiding in the diagnosis through digital image processing may take the form of computer system 600 as reflected in FIG. 6, though variations thereof may readily be implemented by persons skilled in the art as may be desirable for any particular installation. In each such case, one or more computer systems 600 may carry out the foregoing methods as computer code.

Computer system 600 includes a communications bus 602, or other communications infrastructure, which communicates data to other elements of computer system 600. For example, communications bus 602 may communicate data (e.g., text, graphics, video, other data) between bus 602 and an I/O interface 604, which may include a display, a data entry device such as a keyboard, touch screen, mouse, or the like, and any other peripheral devices capable of entering and/or viewing data as may be apparent to those skilled in the art. Further, computer system 600 includes a processor 606, which may comprise a special purpose or a general purpose digital signal processor. Still further, computer system 600 includes a primary memory 608, which may include by way of non-limiting example random access memory (“RAM”), read-only memory (“ROM”), one or more mass storage devices, or any combination of tangible, non-transitory memory. Still further, computer system 600 includes a secondary memory 610, which may comprise a hard disk, a removable data storage unit, or any combination of tangible, non-transitory memory. Finally, computer system 600 may include a communications interface 612, such as a modem, a network interface (e.g., an Ethernet card or cable), a communications port, a PCMCIA slot and card, a wired or wireless communications system (such as Wi-Fi, Bluetooth, Infrared, and the like), local area networks, wide area networks, intranets, and the like.

Each of primary memory 608, secondary memory 610, communications interface 612, and combinations of the foregoing may function as a computer usable storage medium or computer readable storage medium to store and/or access computer software including computer instructions. For example, computer programs or other instructions may be loaded into the computer system 600 such as through a removable data storage device (e.g., a floppy disk, ZIP disks, magnetic tape, portable flash drive, optical disk such as a CD, DVD, or Blu-ray disk, Micro Electro Mechanical Systems (“MEMS”), and the like). Thus, computer software including computer instructions may be transferred from, e.g., a removable storage or hard disc to secondary memory 610, or through data communication bus 602 to primary memory 608.

Communication interface 612 allows software, instructions and data to be transferred between the computer system 600 and external devices or external networks. Software, instructions, and/or data transferred by the communication interface 612 are typically in the form of signals that may be electronic, electromagnetic, optical or other signals capable of being sent and received by communication interface 612. Signals may be sent and received using a cable or wire, fiber optics, telephone line, cellular telephone connection, radio frequency (“RE”) communication, wireless communication, or other communication channels as will occur to those of ordinary skill in the art.

Computer programs, when executed, allow the processor of computer system 600 to implement the methods discussed herein for the diagnoses of a sickle cell disease type from a blood smear image, according to computer software including instructions.

Computer system 600 may perform any one of, or any combination of, the steps of any of the methods described herein. It is also contemplated that the methods according to the present invention may be performed automatically or may be accomplished by some form of manual intervention.

The computer system 600 of FIG. 6 is provided only for purposes of illustration, such that the invention is not limited to this specific embodiment. Persons having ordinary skill in the art are capable of programming and implementing the instant invention using any computer system.

The system of FIG. 1 may, in an exemplary configuration, be implemented in a cloud computing environment for carrying out the methods described herein. That cloud computing environment uses the resources from various networks as a collective virtual computer, where the services and applications can run independently from a particular computer or server configuration making hardware less important. The cloud computer environment includes at least one user computing device. The client computer may be any device that may be used to access a distributed computing environment to perform the methods disclosed herein and may include (by way of non-limiting example) a desktop computer, a portable computer, a mobile phone, a personal digital assistant, a tablet computer, or any similarly configured computing device.

A client computer preferably includes memory such as RAM, ROM, one or more mass storage devices, or any combination of the foregoing. The memory functions as a computer readable storage medium to store and/or access computer software and/or instructions.

A client computer also preferably includes a communications interface, such as a modem, a network interface (e.g., an Ethernet card), a communications port, a PCMCIA slot and card, wired or wireless systems, and the like. The communications interface allows communication through transferred signals between the client computer and external devices including networks such as the Internet and a cloud data center. Communication may be implemented using wireless or wired capability, including (by way of non-limiting example) cable, fiber optics, telephone line, cellular telephone, radio waves or other communications channels as may occur to those skilled in the art.

Such client computer establishes communication with the one or more servers via, for example, the Internet, to in turn establish communication with one or more cloud data centers that implement diagnosing system 100. A cloud data center may include one or more networks that are managed through a cloud management system. Each such network includes resource servers that permit access to a collection of computing resources and components of diagnosing system 100, which computing resources and components can be invoked to instantiate a virtual computer, process, or other resource for a limited or defined duration. For example, one group of resource servers can host and serve an operating system or components thereof to deliver and instantiate a virtual computer. Another group of resource servers can accept requests to host computing cycles or processor time, to supply a defined level of processing power for a virtual computer. Another group of resource servers can host and serve applications to load on an instantiation of a virtual computer, such as an email client, a browser application, a messaging application, or other applications or software.

The cloud management system may comprise a dedicated or centralized server and/or other software, hardware, and network tools to communicate with one or more networks, such as the Internet or other public or private network, and their associated sets of resource servers. The cloud management system may be configured to query and identify the computing resources and components managed by the set of resource servers needed and available for use in the cloud data center. More particularly, the cloud management system may be configured to identify the hardware resources and components such as type and amount of processing power, type and amount of memory, type and amount of storage, type and amount of network bandwidth and the like, of the set of resource servers needed and available for use in the cloud data center. The cloud management system can also be configured to identify the software resources and components, such as type of operating system, application programs, etc., of the set of resource servers needed and available for use in the cloud data center.

In accordance with still further aspects of an embodiment of the invention, a computer program product may be provided to provide software to the cloud computing environment. Computer products store software on any computer useable medium, known now or in the future. Such software, when executed, may implement the methods according to certain embodiments of the invention. By way of non-limiting example, such computer usable mediums may include primary storage devices (e.g., any type of random access memory), secondary storage devices (e.g., hard drives, floppy disks, CD ROMS, ZIP disks, tapes, magnetic storage devices, optical storage devices, MEMS, nanotech storage devices, etc.), and communication mediums (e.g., wired and wireless communications networks, local area networks, wide area networks, intranets, etc.). Those skilled in the art will recognize that the embodiments described herein may be implemented using software, hardware, firmware, or combinations thereof.

The cloud computing environment described above is provided only for purposes of illustration and does not limit the invention to this specific embodiment. It will be appreciated that those skilled in the art are readily able to program and implement the invention using any computer system or network architecture.

Thus and in accordance with all of the foregoing, an integrated decision support system may be provided for the detection of sickle cell disease from blood smear images. Such an integrated system will greatly improve the decision-making process for sickle cell detection/diagnosis. The experimental results indicate that the approach is effective to identify blood smears bearing evidence of sickle cell disease and retrieve visually similar images from a database and to predict the categories of images for diagnostic correctness. Image retrieval and ensemble-based decision making can be integrated and interactively utilized as a diagnostic support tool to help the medical practitioner for detection and diagnosis.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept.

1. An automated method for diagnosing sickle cell disease type from a blood smear image, comprising:
receiving at a processor of a diagnosing system computer a digital query image of a blood smear from a data capture device;
comparing at said processor said digital query image to a plurality of digital images in a database, wherein said database comprises digital blood smear images of pathologically confirmed types of sickle cell disease;
selecting at said processor a plurality of said pathologically confirmed digital images from said database that have a designated similarity to said digital query image; and
causing said processor to display to a user, probabilities that said digital query image displays a blood smear having a pathology matching each of a plurality of sickle cell disease types.

2. The automated method for diagnosing a sickle cell disease type of claim 1, further comprising the step of causing said processor to display said plurality of pathologically confirmed digital images to said user.

3. The automated method for diagnosing a sickle cell disease type of claim 1, wherein said comparing step further comprises applying at said processor a deep feature extraction to said digital query image to generate a feature vector quantifying contents of the digital query image.

4. The automated method for diagnosing a sickle cell disease type of claim 3, wherein said step of applying a deep feature extraction to said digital query image further comprises using at said processor a plurality of pretrained Convolutional Neural Networks feature vectors to generate a combined feature vector.

5. The automated method for diagnosing a sickle cell disease type of claim 3, wherein said comparing step further comprises applying at said processor a classification to said feature vector as one of multiple types of sickle cell disease.

6. The automated method for diagnosing a sickle cell disease type of claim 5, wherein applying a classification to said feature vector further comprising using both Logistical Regression and Support Vector Classifier processes.

7. The automated method for diagnosing a sickle cell disease type of claim 1, further comprising the step of causing said processor to select said plurality of said pathologically confirmed digital images based on a distance measure between a feature vector of said digital query image and said plurality of pathologically confirmed digital images.

8. A system for the automated diagnosing of a sickle cell disease type from a blood smear image, comprising a memory and a processor in data communication with said memory, the memory having computer executable instructions stored thereon configured to be executed by the processor to cause the system to:
receive a digital query image of a blood smear from a data capture device;
compare at said processor said digital query image to a plurality of digital images in a database, wherein said database comprises digital images of blood smears with pathologically confirmed types of sickle cell disease;
select a plurality of said pathologically confirmed digital images from said database that have a designated similarity to said digital query image; and
display to a user, probabilities that said digital query image displays a blood smear having a pathology matching each of a plurality of sickle cell disease types.

9. The system for the automated diagnosing of a sickle cell disease type of claim 8, wherein said computer executable instructions are further configured to cause said processor to display said plurality of pathologically confirmed digital images to said user.

10. The system for the automated diagnosing a sickle cell disease type of claim 8, wherein said computer executable instructions configured to compare said digital query image to the plurality of digital images are further configured to apply a deep feature extraction to said digital query image to generate a feature vector quantifying contents of the digital query image.

11. The system for the automated diagnosing of a sickle cell disease type of claim 10, wherein said computer executable instructions configured to apply a deep feature extraction to said digital query image are further configured to use a plurality of pretrained Convolutional Neural Networks feature vectors to generate a combined feature vector.

12. The system for the automated diagnosing of a sickle cell disease type of claim 10, wherein said computer executable instructions configured to compare said digital query image to the plurality of digital images are further configured to apply a classification to said feature vector as one of multiple types of sickle cell disease.

13. The system for the automated diagnosing of a sickle cell disease type of claim 12, wherein said computer executable instructions configured to apply a classification to said feature vector are further configured to use both Logistical Regression and Support Vector Classifier processes.

14. The system for the automated diagnosing of a sickle cell disease type of claim 8, wherein said computer executable instructions are further configured to select said plurality of said pathologically confirmed digital images based on a distance measure between a feature vector of said digital query image and said plurality of pathologically confirmed digital images.

15. A non-transitory computer-readable medium having stored thereon one or more code sections each comprising a plurality of instructions executable by one or more processors, the instructions configured to cause the one or more processors to perform the actions of an automated method for diagnosing a sickle cell disease type, the actions of the method comprising the steps of:
receiving a digital query image of a blood smear from a data capture device;
comparing said digital query image to a plurality of digital images in a database, wherein said database comprises digital images of blood smears with pathologically confirmed types of sickle cell disease;
selecting a plurality of said pathologically confirmed digital images from said database that have a designated similarity to said digital query image; and
displaying to a user, probabilities that said digital query image displays a blood smear having a pathology matching each of a plurality of sickle cell disease types.

16. The non-transitory computer-readable medium of claim 15, the method further comprising the step of causing said processor to display said plurality of pathologically confirmed digital images to said user.

17. The non-transitory computer-readable medium of claim 15, the method further comprising the step of selecting said plurality of said pathologically confirmed digital images based on a distance measure between a feature vector of said digital query image and said plurality of pathologically confirmed digital images.
